(19) United States
(12) Patent Application Publication
(10) Pub. No.: US 2003/0135023 A1
(43) Pub. Date: Jul. 17, 2003

(54) Peptide structures useful for competitive modulation of dipeptidyl peptidase IV catalysis

(76) Inventors: Hans-Ulrich Demuth, Halle/Saale (DE); Torsten Hoffmann, Halle/Saale (DE); Susanne Manhart, Halle/Saale (DE); Matthias Hoffmann, Wengelsdorf (DE); Jochen Heins, Kurort Hartha (DE)

Correspondence Address: BROWN, RUDNICK, BERLACK & ISRAELS, LLP., BOX IP, 18TH FLOOR, ONE FINANCIAL CENTER, BOSTON, MA, 02111, US

(21) Appl. No.: 10/186,177

(22) Filed: Jun. 27, 2002

Related U.S. Application Data
(60) Provisional application No. 60/301,158, filed on Jun. 27, 2001.

(51) Int. Cl. C07K 7/06; C07K 5/06

(52) U.S. Cl. 530/329; 530/330

(57) ABSTRACT

This invention involves a compound represented by the general formula (I):

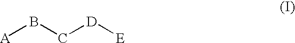

and pharmaceutically acceptable salts thereof,

wherein

A is any amino acid except a D-amino acid;

B is an amino acid selected from Pro, Ala, Ser, Gly, Hyp, acetidine-(2)-carboxylic acid and pipecolic acid,

C is any amino acid except Pro, Hyp, acetidine-(2)-carboxylic acid, pipecolic acid and except N-alkylated amino acids, e.g. N-methyl valine and sarcosine,

D is any amino acid or missing, and

E is any amino acid or missing;

or

wherein

C is any amino acid except Pro, Hyp, acetidine-(2)-carboxylic acid, pipecolic acid, except N-alkylated amino acids, e.g. N-methyl valine and sarcosine and except a D-amino acid,

D is an amino acid selected from Pro, Ala, Ser, Gly, Hyp, acetidine-(2)-carboxylic acid and pipecolic acid, and

E is any amino acid except Pro, Hyp, acetidine-(2)-carboxylic acid, pipecolic acid and except N-alkylated amino acids, e.g. N-methyl valine and sarcosine and methods of manufacture and use.

### RELATED APPLICATIONS

This application claims the benefit from U.S. Provisional application Ser. No. 60/301,158 filed Jun. 27, 2001 which is incorporated herein by reference in its entirety.

### BACKGROUND OF THE INVENTION

1. Field of the Invention

The present invention relates to the function of dipeptidyl peptidase IV (DP IV, synonym: DPP IV, CD26, EC 3.4.14.5) and DP IV-like enzymes within a subject and their biological effects on the plasma levels of the insulinotropic peptides gastric inhibitory polypeptide 1-42 (GIP₁₋₄₂) and glucagon-like peptide amides-1 (GLP-1₇₋₃₆) and (GLP-1₇₋₃₇) or analogues thereof. The invention relates further to the treatment of impaired glucose tolerance, diabetes mellitus, glucosuria and metabolic acidosis by selective modulation of the activity of DP IV-like enzymes due to the use of tri-, tetra- and pentapeptide substrates of dipeptidyl peptidase IV in pharmacological doses to inhibit the physiological turnover of endogenous peptide hormones.

2. Background of the Invention

Dipeptidyl peptidase IV (DP IV) is a serine protease which cleaves off N-terminal dipeptides from a peptide chain containing, preferably a proline residue in the penultimate position.

DP IV-like enzymes are structurally related enzymes to DP IV (Blanco et. al., 1998) which may share a certain sequence homology to the DP IV sequence, but which share even if they are not structurally related (by convergent evolution) the substrate specificity of DP IV of removing dipeptides from the N-termini of polypeptides by cleaving after a penultimate proline residue. Such enzymes—including DP IV, DP II at one hand and attractin on the other hand (Fukasawa et al., 2001)—are also capable to remove dipeptides with a penultimate alanine (or serine or glycine residues) from the N-termini of polypeptides but usually with reduced catalytic efficacy as compared to the post-proline cleavage (Yaron & Naider, 1993). They show the common feature that they accommodate in the Pro-position of the target-protein also Ala, Ser, Thr and other amino acids with small hydrophobic side-chains as, Gly or Val. The hydrolytic efficacy is ranked Pro>Ala>>Ser, Thr >>Gly, Val. While the proteins DPIV, DP II, FAPα (Seprase), DP 6, DP 8 and DP 9 are structurally related and show a high sequence homology, attractin is an extraordinary functional DPIV-like enzyme (Sedo & Malik, 2001).

Further DPIV-like enzymes are disclosed in WO 01/19866, WO 02/04610, WO 02/34900 and WO 02/31134. WO 01/19866 discloses human dipeptidyl aminopeptidase 8 (DPP8) with structural und functional similarities to DPIV and fibroblast activation protein (FAP). The dipeptidyl peptidase IV-like enzyme of WO 02/04610 is well known in the art. In the GENE BANK data base, this enzyme is registered as KIAA1492 (registration in February 2001, submitted on Apr. 04, 2000, AB040925) and in the MEROPS data base. WO 02/34900 discloses a dipeptidyl peptidase 9 (DPP9) with significant homology to the amino acid sequences of DPIV and DPP8. WO 02/31134 discloses three DPIV-like enzymes, DPRP1, DPRP2 and DPRP3. Sequence analysis revealed that DPRP1 is identical to DPP8, as disclosed in WO 01/19866, that DPRP2 is identical to DPP9 and that DPRP3 is identical to KIAA1492 as disclosed in WO 02/04610.

More recently, it was shown that DP IV is responsible for cleaving glucagon-like peptide-1 and gastric inhibitory peptides, thereby shortening the half life of GLP-1 and GIP and their physiological response in the circulation. From inhibition of serum DP IV, a significant increase in the bioactivity of the incretins has been shown. Since the incretins are major stimulators of pancreatic insulin secretion and have direct beneficial effects on glucose disposal, DP IV inhibition represents an attractive approach for treating impaired glucose tolerance and non-insulin-dependent diabetes mellitus (NIDDM) and related disorders, like glucosuria and metabolic acidosis (see DE 196 16 486 and WO 97/40832).

The substrate specificity of the enzyme dipeptidyl peptidase IV, may be summarized in the following way:

1. Dipeptidyl peptidase IV hydrolyzes oligopeptides and proteins from the N-terminus, splitting off dipeptide units, when the penultimate residue is proline, hydroxyproline, dehydroproline, pipecolic acid or alanine. The best substrates according to their k_{cat}/Kₘ values are those with a proline residue in the P1-position.

2. DP IV requires a ‘trans’ peptide-bond between P1 and P2 residues.

3. The N-terminal amino group of substrates must be protonated in order to be susceptible to DP IV.

4. A proline residue in the P1′-position of substrates prevents substrate hydrolysis by dipeptidyl peptidase IV. This enzyme does not release arginylproline from bradykinin, for instance.

### SUMMARY OF THE INVENTION

The present invention is directed to compounds represented by formula (I),

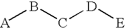

with certain restrictions as detailed hereafter.

These compounds are substrates of proline-specific peptidases, in particular of DP IV and other enzymes having similar DP IV-like enzymatic activity profiles (“DP IV-like enzymes”), and may be useful either as substrates or as antagonists of DP IV and DP IV-like enzymes to inhibit the physiological turnover of endogenous peptide hormones by competitive catalysis.

The compounds of formula (I) may be used for treating impaired glucose tolerance, diabetes mellitus, glucosuria, and metabolic acidosis diagnosed in a subject.

### BRIEF DESCRIPTION OF THE DRAWINGS

FIG. 1 shows plasma DP IV activity after intravasal administration of 10, 30 and 100 mg/kg b.w. Ile-Pro-le in Wistar rats;

FIG. 2 shows plasma DP IV activity after administration of 10, 30 and 100 mg/kg b.w. Ile-Pro-Ile and of 10 mg/kg b.w. and isoleucyl thiazolidine fumarate as positive control in Wistar rats (AUC 0-20 min);

FIG. 3 shows plasma DP IV activity after intravasal administration of 10, 30 and 100 mg/kg b.w. Val-Pro-Leu in Wistar rats;

FIG. 4 shows plasma DP IV activity after administration of 10, 30 and 100 mg/kg b.w. Val-Pro-Leu and of 10 mg/kg b.w. isoleucyl thiazolidine fumarate as positive control in Wistar rats (AUC 0-20 min);

FIG. 5 shows plasma DP IV activity after oral and intravasal administration of 100 mg/kg b.w. t-butyl-Gly-Pro-Ile in Wistar rats;

FIG. 6 shows plasma DP IV activity after oral and intravasal administration of 100 mg/kg b.w. t-butyl-Gly-Pro-Ile, and of 10 mg/kg b.w. isoleucyl thiazolidine fumarate as positive control in Wistar rats(AUC 0-120 min);

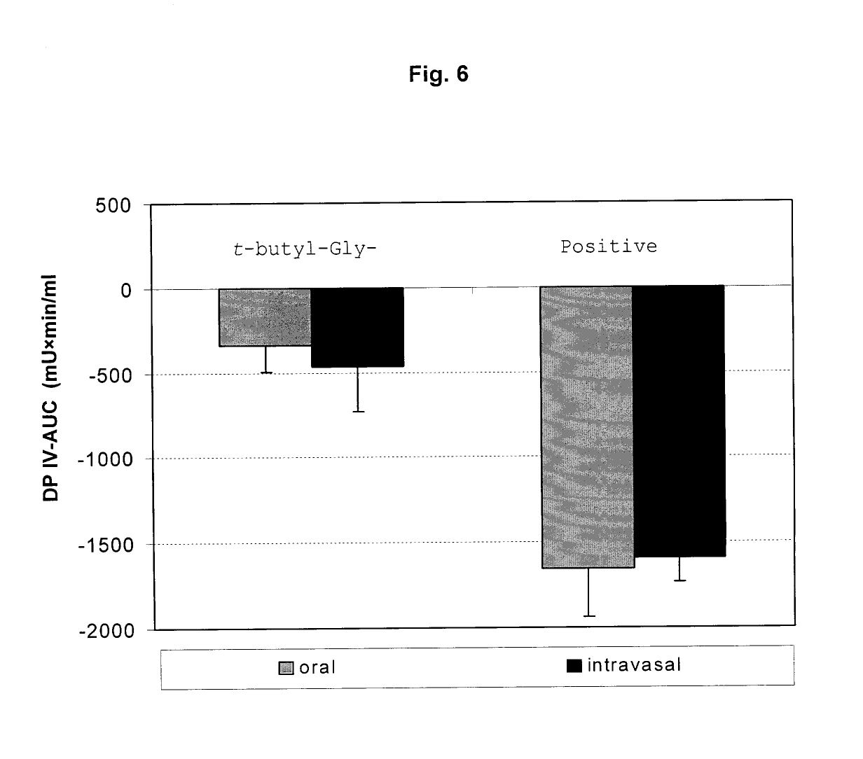

FIG. 7 shows the course of plasma glucose concentration after oral administration of 100 mg/kg b.w. t-butyl-Gly-Pro-Ile, and of 10 mg/kg b.w. isoleucyl thiazolidine fumarate as positive control in diabetic Zucker rats; and

FIG. 8 shows the improvement of glucose tolerance and G-AUC during OGTT after oral administration of 100 mg/kg b.w. t-butyl-Gly-Pro-Ile, and of 10 mg/kg b.w. isoleucyl thiazolidine fumarate as positive control diabetic Zucker rats (G-AUC 0-60 min).

### DETAILED DESCRIPTION OF THE INVENTION

More particularly, the present invention is directed to peptides of the following formula (I):

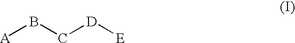

wherein

A, B, C, D and E are any amino acids including proteinogenic amino acids, non-proteinogenic amino acids, L-amino acids and D-amino acids and wherein E and/or D may be absent or B and/or A may be absent with additional conditions as hereinafter detailed:

Further conditions regarding formula (I):

A is any amino acid residue except a D-amino acid;

B is an amino acid selected from Pro, Ala, Ser, Gly, Hyp, acetidine-(2)-carboxylic acid and pipecolic acid,

C is any amino acid except Pro, Hyp, acetidine-(2)-carboxylic acid, pipecolic acid and except N-alkylated amino acids, e.g. N-methyl valine and sarcosine,,

D is any amino acid or missing, and

E is any amino acid or missing

or

C is any amino acid except Pro, Hyp, acetidine-(2)-carboxylic acid, pipecolic acid, except N-alkylated amino acids, e.g. N-methyl valine and sarcosine and except a D-amino acid,,

D is an amino acid selected from Pro, Ala, Ser, Gly, Hyp, acetidine-(2)-carboxylic acid and pipecolic acid, and

E is any amino acid except Pro, Hyp, acetidine-(2)-carboxylic acid, pipecolic acid and except N-alkylated amino acids, e.g. N-methyl valine and sarcosine..

The present invention especially refers to compounds of formula (I)

wherein

A is any amino acid except a D-amino acid;

B is an amino acid selected from Pro, Ala, Ser, Gly, Hyp, acetidine-(2)-carboxylic acid and pipecolic acid,

C is any amino acid except Pro, Hyp, acetidine-(2)-carboxylic acid, pipecolic acid and except N-alkylated amino acids, e.g. N-methyl valine and sarcosine,,

D is any amino acid or missing, and

E is any amino acid or missing.

The present invention moreover refers to compounds of formula (I)

wherein

A is any amino acid except a D-amino acid;

B is an amino acid selected from Pro, Ala, Ser, Gly, Hyp, acetidine-(2)-carboxylic acid and pipecolic acid,

C is any amino acid except Pro, Hyp, acetidine-(2)-carboxylic acid, pipecolic acid, except N-alkylated amino acids, e.g. N-methyl valine and sarcosine and except a D-amino acid,

D is an amino acid selected from Pro, Ala, Ser, Gly, Hyp, acetidine-(2)-carboxylic acid and pipecolic acid, and

E is any amino acid except from Pro, Hyp, acetidine-(2)-carboxylic acid, pipecolic acid and except N-alkylated amino acids, e.g. N-methyl valine and sarcosine.

Preferred

A is a L-amino acid.

Further preferred

C is a L-amino acid;

further preferred

E is a missing;

further preferred

D and E are missing;

further preferred

A is t-butyl-Gly, lie or Val;

especially preferred

A is t-butyl-Gly;

further preferred

B is Pro;

further preferred

D is Pro;

further preferred

C is t-butyl-Gly, Ile or Val;

more preferred

C is t-butyl-Gly or Val;

especially preferred

C is t-butyl-Gly;

especially preferred are

t-butyl-Gly-Pro-Ile; t-butyl-Gly-Pro-Val; Val-Pro-t-butyl-Gly, Ile-Pro-t-butyl-Gly or t-butyl-Gly-Pro-t-butyl-Gly and pharmaceutically acceptable salts thereof.

The compound of the present invention can be in the free acid peptide form or the C-terminal amide peptide form.

The compounds of the present invention may be present as the free C-terminal acid or as the C-terminal amide form. The free acid peptides or the amides may be varied by side chain modifications. Such side chain modifications are for instance, but not restricted to, homoserine addition, pyroglutamic acid addition, disulphide bond formation, deamidation of asparagine or glutamine residues, methylation, t-butylation, t-butyloxycarbonylation, 4-methylbenzylation, thioanysilation, thiocresylation, benzyloxymethylation, 4-nitrophenylation, benzyloxycarbonylation, 2-nitrobencoylation, 2-nitrosulphenylation, 4-toluenesulphonylation, pentafluorophenylation, diphenylmethylation, 2-chlorobenzyloxycarbonylation, 2,4,5-trichlorophenylation, 2-bromobenzyloxycarbonylation, 9-fluorenylmethyloxycarbonylation, triphenylmethylation, 2,2,5,7,8, -pentamethylchroman-6-sulphonylation, hydroxylation, oxidation of methionine, formylation, acetylation, anisylation, benzylation, benzoylation, trifluoroacetylation, carboxylation of aspartic acid or glutamic acid, phosphorylation, sulphation, cysteinylation, glycolysation with pentoses, deoxyhexoses, hexosamines, hexoses or N-acetylhexosamines, farnesylation, myristolysation, biotinylation, palmitoylation, stearoylation, geranylgeranylation, glutathionylation, 5′-adenosylation, ADP-ribosylation, modification with N-glycolylneuraminic acid, N-acetylneuraminic acid, pyridoxal phosphate, lipoic acid, 4′-phosphopantetheine, or N-hydroxysuccinimide.

In the compounds of formula (I), the amino acids A, B, C, D, and E, respectively, are attached to the adjacent amino acid with amide bonds in a usual manner and according to standard nomenclature so that the amino-terminus (N-terminus) of the amino acids is drawn on the left and the carboxyl-terminus of the amino acid is drawn on the right.

Examples of amino acids which can be used in the present invention are L and D-amino acids, N-methyl-amino-acids; allo- and threo-forms of lie and Thr, which can, e.g. be α-, β- or ω-amino acids, whereof α-amino acids are preferred.

Examples of amino acids are:

aspartic acid (Asp), glutamic acid (Glu), arginine (Arg), lysine (Lys), histidine (His), glycine (Gly), serine (Ser) and cysteine (Cys), threonine (Thr), asparagine (Asn), glutamine (Gin), tyrosine (Tyr), alanine (Ala), proline (Pro), valine (Val), isoleucine (lie), leucine (Leu), methionine (Met), phenylalanine (Phe), tryptophan (Trp), hydroxyproline (Hyp), beta-alanine (beta-Ala), 2-amino octanoic acid (Aoa), azetidine-(2)-carboxylic acid (Ace), pipecolic acid (Pip), 3-amino propionic, 4-amino butyric and so forth, alpha-aminoisobutyric acid (Aib), sarcosine (Sar), ornithine (Orn), citrulline (Cit), homoarginine (Har), t-butylalanine (t-butyl-Ala), t-butylglycine (t-butyl-Gly), N-methylisoleucine (N-Melle), phenylglycine (Phg), cyclohexylalanine (Cha), norleucine (Nle), cysteic acid (Cya) and methionine sulfoxide (MSO), Acetyl-Lys, modified amino acids such as phosphoryl-serine (Ser(P)), benzyl-serine (Ser(Bzl)) and phosphoryl-tyrosine (Tyr(P)), 2-aminobutyric acid (Abu), aminoethylcysteine (AECys), carboxymethylcysteine (Cmc), dehydroalanine (Dha), dehydroamino-2-butyric acid (Dhb), carboxyglutaminic acid (Gla), homoserine (Hse), hydroxylysine (Hyl), cis-hydroxyproline (cisHyp), trans-hydroxyproline (transHyp), isovaline (Iva), pyroglutamic acid (Pyr), norvaline (Nva), 2-aminobenzoic acid (2-Abz), 3-aminobenzoic acid (3-Abz), 4- aminobenzoic acid (4-Abz), 4-(aminomethyl)benzoic acid (Amb), 4-(aminomethyl)cyclohexanecarboxylic acid (4-Amc), Penicillamine (Pen), 2-Amino-4-cyanobutyric acid (Cba), cycloalkane-carboxylic aicds.

Examples of \overline{ω}-amino acids are e.g.: 5-Ara (aminoraleric acid), 6-Ahx (aminohexanoic acid), 8-Aoc (aminooctanoic aicd), 9-Anc (aminovanoic aicd), 10-Adc (aminodecanoic acid), 11-Aun (aminoundecanoic acid), 12-Ado (aminododecanoic acid).

Further amino acids are: indanylglycine (Igl), indoline-2-carboxylic acid (Idc), octahydroindole-2-carboxylic acid (Oic), diaminopropionic acid (Dpr), diaminobutyric acid (Dbu), naphtylalanine (1-Nal), (2-Nal), 4-aminophenylalanin (Phe(4 -NH₂)), 4-benzoylphenylalanine (Bpa), diphenylalanine (Dip), 4 -bromophenylalanine (Phe(4-Br)), 2-chlorophenylalanine (Phe(2-Cl)), 3-chlorophenylalanine (Phe(3-Cl)), 4-chlorophenylalanine (Phe(4-Cl)), 3,4-chlorophenylalanine (Phe (3,4-Cl₂)), 3 - fluorophenylalanine (Phe(3-F)), 4-fluorophenylalanine (Phe(4-F)), 3,4- fluorophenylalanine (Phe(3,4-F₂)), pentafluorophenylalanine (Phe(F₅)), 4-guanidinophenylalanine (Phe(4 -guanidino)), homophenylalanine (hPhe), 3-jodophenylalanine (Phe(3-J)), 4 jodophenylalanine (Phe(4-J)), 4-methylphenylalanine (Phe(4-Me)), 4-nitrophenylalanine (Phe-4-NO₂)), biphenylalanine (Bip), 4-phosphonomehtylphenylalanine (Pmp), cyclohexyglycine (Ghg), 3-pyridinylalanine (3-Pal), 4-pyridinylalanine (4-Pal), 3,4-dehydroproline (A-Pro), 4-ketoproline (Pro(4-keto)), thioproline (Thz), isonipecotic acid (Inp), 1,2,3,4,-tetrahydroisoquinolin-3-carboxylic acid (Tic), propargylglycine (Pra), 6-hydroxynorleucine (NU(6-OH)), homotyrosine (hTyr), 3-jodotyrosine (Tyr(3-J)), 3,5-dijodotyrosine (Tyr(3,5-J₂)), d-methyl-tyrosine (Tyr(Me)), 3-NO₂-tyrosine (Tyr(3-NO₂)), phosphotyrosine (Tyr(PO₃H₂)), alkylglycine, 1-aminoindane-1-carboxy acid, 2-aminoindane-2 -carboxy acid (Aic), 4-amino-methylpyrrol-2-carboxylic acid (Py), 4 -amino-pyrrolidine-2-carboxylic acid (Abpc), 2-aminotetraline-2 -carboxylic acid (Atc), diaminoacetic acid (Gly(NH₂)), diaminobutyric acid (Dab), 1,3-dihydro-2H-isoinole-carboxylic acid (Disc), homocylcohexylalanin (hCha), homophenylalanin (hPhe oder Hof), trans-3-phenyl-azetidine-2-carboxylic acid, 4-phenyl-pyrrolidine-2-carboxylic acid, 5-phenyl-pyrrolidine-2 -carboxylic acid, 3-pyridylalanine (3-Pya), 4-pyridylalanine (4 -Pya), styrylalanine, tetrahydroisoquinoline-1-carboxylic acid (Tiq), 1,2,3,4-tetrahydronorharmane-3-carboxylic acid (Tpi), β-(2 -thienryl)-alanine (Tha)

Other amino acid substitutions for those encoded in the genetic code can also be included in peptide compounds within the scope of the invention.

The present invention furthermore refers to a pharmaceutical composition comprising at least one compound of the present invention and a pharmaceutically acceptable carrier and/or diluent.

Such pharmaceutical compositions can be prepared by mixing at least one compound of the present invention and a pharmaceutically acceptable carrier and/or diluent.

The compounds and compositions according to the present invention can be used for the preparation of a medicament for the prophylaxis or treatment of a condition mediated by modulation of the dipeptidyl peptidase IV activity.

Such conditions are, e.g. selected from impaired glucose tolerance, diabetes mellitus, glucosuria and metabolic acidosis.

The term “subject” as used herein, refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human, being sought by a researcher, veterinarian, medical doctor or other clinician, which includes alleviation of the symptoms of the disease or disorder being treated.

As used herein, the term “composition” is intended to encompass a product comprising at least one of the compounds of the present invention in the therapeutically effective amounts, as well as any product which results, directly or indirectly, from combinations of the claimed compounds.

The compounds of the present invention may also be present in the form of a pharmaceutically acceptable salt. The pharmaceutically acceptable salt generally takes a form in which an amino acids basic side chain is protonated with an inorganic or organic acid. Representative organic or inorganic acids include, e.g. hydrochloric, hydrobromic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toulenesulfonic, cyclohexanesulfamic, salicylic, saccharinic or trifluoroacetic acid.

The present invention further includes within its scope prodrugs of the compounds of this invention. In general, such prodrugs will be functional derivatives of the compounds which are readily convertible in vivo into the desired therapeutically active compound. Thus, in these cases, the use of the present invention shall encompass the treatment of the various disorders described with prodrug versions of one or more of the claimed compounds, but which converts to the above specified compound in vivo after administration to the subject. Conventional procedures for the selection and preparation of suitable prodrug derivatives are described, for example, in “Design of Prodrugs”, ed. H. Bundgaard, Elsevier, 1985 and the patent applications DE 198 28 113; WO 99/67278, DE 198 28 114 and WO 99/67279, fully incorporated herein by reference.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms of the compounds may exist as polymorphs and as such are intended to be included in the present invention. In addition, some of the compounds may form solvates with water (i.e. hydrates) or common organic solvents, and such solvates are also intended to be encompassed within the scope of this invention.

Until the present invention by Applicants, known peptide substrates of the proline-specific serine protease dipeptidyl peptidase IV in vitro are the tripeptides Diprotin A (Ile-Pro-Ile), Diprotin B (Val-Pro-Leu) and Diprotin C (Val-Pro-Ile). These compounds per se are excluded from the present invention. Applicants have unexpectedly discovered that the compounds disclosed here act as substrates of dipeptidyl peptidase IV in vivo and, in pharmacological doses, inhibit the physiological turnover of endogenous peptide hormones by competitive catalysis.

Particularly preferred compounds or prodrugs of the present invention that could be useful as modulators of dipeptidyl peptidase IV and DP IV—like enzymes include those compounds or prodrugs which show Kᵢ-values for DP IV binding, effectivity in DP IV inhibition in vivo after intravasal (i.v.) and/or oral (p.o.) administration to Wistar rats and improved glucose tolerance in vivo after i.v. and p.o. administration to fa/fa Zucker rats.

The modulators of this invention may be prepared using solid phase chemistry or, alternatively, via normal solution chemistry, using conventional methods known in the art.

The utility of the compounds of formula (I) to act as DP IV substrates to inhibit the physiological turnover of endogenous peptide hormones by competitive catalysis in vivo can be determined according to the procedures described in Examples 3 and 4. The present invention therefore provides a method of preventing or treating a condition mediated by modulation of the DP IV activity in a subject in need thereof which comprises administering any of the compounds or pharmaceutical compositions thereof in a quantity and dosing regimen therapeutically effective to treat the condition. Additionally, the present invention includes the use of a compound of formula (I) for the preparation of a medicament for the prevention or treatment of a condition mediated by modulation of the DP IV activity in a subject. The compound may be administered to a patient by any conventional route of administration, including, but not limited to, intravenous, oral, subcutaneous, intramuscular, intradermal and parenteral or combinations thereof. Oral administration is preferred.

The present invention also provides pharmaceutical compositions comprising one or more compounds of this invention in association with a pharmaceutically acceptable carrier and/or diluent.

To prepare the pharmaceutical compositions of this invention, one or more compounds of formula (I) or salts thereof as the active ingredients, are intimately admixed with a pharmaceutical carrier and/or diluent according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives may advantageously include water, glycols, oils, alcohols, flavoring agents, preservatives, coloring agents and the like; for solid oral preparations such as, for example, powders, capsules, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are employed. If desired, tablets may be sugar coated or enteric coated by standard techniques. For parenterals, the carrier will usually comprise sterile water, through other ingredients, for example, for purposes such as aiding solubility or for preservation, may be included.

Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per dosage unit, e.g., tablet, capsule, powder, injection, suppository, teaspoonful and the like, of from about 0.01 mg to about 1000 mg (preferably about 5 to about 500 mg) and may be given at a dosage of from about 0.1 to about 300 mg/ kg bodyweight per day (preferably 1 to 50mg/kg per day). The dosages, however, may be varied depending upon the requirement of the patients, the severity of the condition being treated and the compound being employed. The use of either daily administration or post-periodic dosing may be employed. Typically the dosage will be regulated by the physician based on the characteristics of the patient, his/her condition and the therapeutic effect desired.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral, parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is ideally mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is ideally dispersed evenly throughout the composition so that the composition may be readily subdivided into equally effective dosage forms such as tablets, pills and capsules. This solid preformulation composition may then be subdivided into unit dosage forms of the type described above containing from 0.01 to about 1000 mg, preferably from about 5 to about 500 mg of the active ingredient of the present invention.

The tablets or pills of the novel composition can be advantageously coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids with such materials as shellac, cetyl alcohol and cellulose acetate.

The liquid forms in which the novel compositions of the present invention may be advantageously incorporated for administration orally or by injection include aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

Where the processes for the preparation of the compounds according to the invention give rise to a mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their components enantiomers by standard techniques, such as the formation of diastereomeric pairs by salt formation with an optically active acid, such as (−)-di-p-toluoyl-d-tartaric acid and/or (+)-di-p-toluoyl-l-tartaric acid followed by fractional crystallization and regeneration of the free base. The compounds may also resolved by formation of diastereomeric esters or amides, followed by chromatographic separation and removal of the chiral auxiliary. Alternatively, the compounds may be resolved using a chiral HPLC column.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991, fully incorporated herein by reference. The protecting groups may be removed at a convenient subsequent stage using methods known from the art.

The method of treating conditions modulated by dipetidyl peptidase IV and DP IV-like enzymes described in the present invention may also be carried out using a pharmaceutical composition comprising one or more of the compounds as defined herein and a pharmaceutically acceptable carrier. The pharmaceutical composition may contain between about 0.0 1 mg and 1000 mg, preferably about 5 to about 500 or 250 mg of the compounds, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of transdermal delivery system, the dosage administration will, of course, be continuous rather than intermittent throughout the dosage regimen and dosage strength will need to be accordingly modified to obtain the desired therapeutic effects.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or betalactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitable flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

The compound of the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine or phosphatidylcholines using processes well described in the art.

Compounds of the present invention may also be delivered by the use of antibodies, most preferably monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamidephenol, polyhydroxyethylaspartamidephenol, or polyethyl eneoxidepolyllysine substituted with palmitoyl residue. Furthermore, the compounds of the present invention may be coupled to a class of biodegradable polymers useful in achieving controlled release of a drug, for example, polyactic acid, polyepsilon caprolactone, polyhydroxy butyeric acid, polyorthoesters, polyacetals, polydihydropyrans, polycyanoacrylates and cross-linked or amphipathic block copolymers of hydrogels.

Compounds of this invention may be administered in any of the foregoing compositions and according to dosage regimens established in the art whenever treatment of the addressed disorders is required.

The daily dosage of the products may be varied over a wide range from 0.01 to 1.000 mg per adult human per day. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250, 500 and 1000 milligrams of the active ingredient for the symptomatic adjustment of the dosage to the patient to be treated. An effective amount of the drug is ordinarily supplied at a dosage level of from about 0.1 mg/kg to about 300 mg/kg of body weight per day. Preferably, the range is from about 1 to about 50 mg/kg of body weight per day. The compounds may be administered on a regimen of 1 to 4 times per day.

Optimal dosages to be administered may be readily determined by those skilled in the art, and will vary with the particular compound used, the mode of administration, the strength of the preparation, bioavailability due to the mode of administration, and the advancement of disease condition. In addition, factors associated with the particular patient being treated, including patient age, weight, diet and time of administration, should generally be considered in adjusting dosages.

The compounds or compositions of the present invention may be taken before a meal, while taking a meal or after a meal.

When taken before a meal, the compounds or compositions of the present invention can be taken 1 hour, preferably 30 or even 15 or 5 minutes before eating.

When taken while eating, the compounds or compositions of the present invention can be mixed into the meal or taken in a separate dosage form as described above.

When taken after a meal, the compounds and compositions of the present invention can be taken 5, 15, or 30 minutes or even 1 hour after finishing a meal.

### EXAMPLES OF THE INVENTION

### Example 1

### Synthesis of Xaa-Pro-Yaa Tripeptides

General procedure

All syntheses were carried out on a peptide synthesizer SP 650 (Labortec AG) applying Fmoc/tBu-strategy. Protected amino acids were purchased from Novabiochem or Bachem. Trifluoro acetic acid (TFA) was purchased from Merck, triisopropyl silane (TIS) was purchased from Fluka.

Pre-loaded Fmoc-Yaa-Wang resin (2.8 g/ substitution level 0.57 mmol/g) was deprotected using 20% piperidine/ N,N-dimethylformamide (DMF). After washing with DMF a solution of 2 eq (1.1 g) of Fmoc-Pro-OH were solved in DMF (12 ml solvent per gram resin). 2eq (1.04 g) of 2-(1 H-Benzotriazole 1-yl)-1,1,3,3-tetramethyluronium tetrafluoroborate (TBTU) and 4 eq (1.11 ml) of N,N-diisopropylethylamine (DIEA) were added and placed in the reaction vessel. The mixture was shaken at room temperature for 20 minutes. Then, the coupling cycle was repeated. After subsequent washing with DMF, dichlormethane, isopropanol and diethyl ether the resulting Fmoc-Pro-Ile-Wang resin was dried and divided into 6 parts before coupling the last amino acid derivative.

Fmoc protecting group was removed as described above. After that 0.54 mmol of the Boc-amino acid, 0.54 mmol TBTU and 0.108 mmol DIEA in DMF were shaken for 20 min. The coupling cycle was repeated. Finally the peptide resin was washed and dried described above.

The peptide was cleaved from the resin using a mixture of trifluoroacetic acid (TFA) for 2.5 h, containing the following scavengers: TFA/H₂O/triisipropylsilane (TIS)=9.5/0.25/0.25 25

The yields of crude peptides were 80-90% on the average. The crude peptides were purified by HPLC on a Nucleosil C18 column (7 μm, 250*21.20 mm, 100 A) using a linear gradient of 0.1% TFA/H₂O with increasing concentration of 0.1% TFA/acetonitrile (from 5% to 65% in 40 min) at 6 ml/min.

The pure peptides were obtained by lyophilization, identified by Electrospray mass spectrometry and HPLC analysis.

### Results

TABLE 1
Identification of Xaa-Pro-Yaa
tripeptides after chemical synthesis

| | | Mass (exp.)¹ | |
|---|---|---|---|
| Peptide | Mass (calc.) | [M + H⁺] | HPLC K′² |
| 2-Amino octanoic acid- | 369.5 | 370.2 | 10.63 |
| Pro-Ile | | | |
| Abu-Pro-Ile | 313.4 | 314.0 | 5.7 |
| Aib-Pro-Ile | 313.4 | 314.0 | 5.25 |
| Aze-Pro-Ile | 311.4 | 312.4 | 5.29 |
| Cha-Pro-Ile | 381.52 | 382.0 | 10.4 |
| Ile-Hyp-Ile | 356.45 | 358.2 | 6.57 |
| Ile-Pro-allo-Ile | 341.4 | 342.0 | 7.72 |
| Ile-Pro-t-butyl-Gly | 341.47 | 342.36 | 6.93 |
| Ile-Pro-Val | 327.43 | 328.5 | 6.41 |
| Nle-Pro-Ile | 341.45 | 342.2 | 8.09 |
| Nva-Pro-Ile | 327.43 | 328.2 | 6.82 |
| Orn-Pro-Ile | 342.42 | 343.1 | 3.73 |
| Phe-Pro-Ile | 375.47 | 376.2 | 8.96 |
| Phg-Pro-Ile | 361.44 | 362.2 | 7.90 |
| Pip-Pro-Ile | 338.56 | 340.0 | 6.50 |
| Ser(Bzl)-Pro-Ile | 405.49 | 406.0 | 9.87 |
| Ser(P)-Pro-Ile | 395.37 | 396.0 | 3.35 |
| Ser-Pro-Ile | 315.37 | 316.3 | 5.24 |
| t-butyl-Gly-Pro-D-Val | 327.4 | 328.6 | 7.27 |
| t-butyl-Gly-Pro-Gly | 285.4 | 286.3 | 3.74 |
| t-butyl-Gly-Pro-Ile | 341.47 | 342.1 | 7.16 |
| t-butyl-Gly-Pro-Ile-amide | 340.47 | 341.3 | 7.8 |
| t-butyl-Gly-Pro-t- | 341.24 | 342.5 | 9.09 |
| butyl-Gly | | | |
| t-butyl-Gly-Pro-Val | 327.4 | 328.4 | 6.32 |
| Thr-Pro-Ile | 329.4 | 330.0 | 5.12 |
| Tic-Pro-Ile | 387.46 | 388.0 | 8.57 |
| Trp-Pro-Ile | 414.51 | 415.2 | 9.85 |
| Tyr(P)-Pro-Ile | 471.47 | 472.3 | 5.14 |
| Tyr-Pro-allo-Ile | 391.5 | 392.0 | 7.02 |
| Val-Pro-allo-Ile | 327.4 | 328.5 | 6.51 |
| Val-Pro-t-butyl-Gly | 327.4 | 328.15 | 5.98 |
| Val-Pro-Val | 313.4 | 314.0 | 5.07 |

¹[M + H⁺] were determined by Electrospray mass spectrometry in positive ionization mode.
²RP-HPLC conditions:
column: LiChrospher 100 RP 18 (5 μm), 125 × 4 mm
detection (UV): 214 nm
gradient system: acetonitrile (ACN)/H₂O (0.1% TFA) from 5% ACN to 50% in 15 min, flow 1 ml/min
k′ = (tᵣ − t₀)/t₀
t₀ = 1.16 min

t-butyl-Gly is defined as:

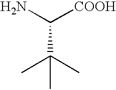

Ser(Bzl) and Ser(P) are defined as benzyl-serine and phosphoryl-serine, respectively. Tyr(P) is defined as phosphoryl-tyrosine.

### Example 2

### Determination of IC₅₀- and Kᵢ-Values of Xaa-Pro-Yaa Tripeptides

### Methods

Determination of IC₅₀-Values

100 μl inhibitor stock solution were mixed with 100 μl buffer (HEPES pH 7.6) and 50 μl substrate (Gly-Pro-pNA, final concentration 0.4 mM) and preincubated at 30° C. Reaction was started by addition of 20 μl purified porcine DP IV. Formation of the product pNA was measured at 405 nm over 10 min using the HTS 7000 Plus plate reader (Perkin Elmer) and slopes were calculated. The final inhibitor concentrations ranged between 1 mM and 30 nM.

For calculation of IC₅₀-values GraFit 4.0.13 (Erithacus Software) was used.

Determination of Kᵢ-Values

For determination of the Kᵢ-values DP IV activity was measured in the same way as described above at final substrate concentrations of 0.05, 0.1, 0.2, and 0.4 mM and further 7 inhibitor concentrations covering the IC₅₀ concentration. Calculations were performed using the GraFit Software.

### Results

TABLE 2
IC₅₀-values of Xaa-Pro-Yaa tripeptides

| Compound | IC₅₀ (mol/l) | SD (mol/l) |
|---|---|---|
| Abu-Pro-Ile | 3.43e−5 | 1.75e−6 |
| Aib-Pro-Ile | no inhibition | |
| AOA-Pro-Ile | 4.21e−5 | 1.26e−6 |
| Aze−Pro-Ile | 7.28e−5 | 5.00e−6 |
| Cha-Pro-Ile | 2.03e−5 | 2.12e−7 |
| Diprotin A | 4.69e−6 | 4.11e−7 |
| Diprotin B | 5.54e−5 | 5.49e−6 |
| Ile−Hyp-Ile | 6.00e−3 | 6.80e−4 |
| Ile−Pro-(allo)Ile | 1.54e−5 | 3.81e−7 |
| Ile−Pro-t-butyl-Gly | 8.23e−5 | 3.84e−6 |
| Ile−Pro-Val | 1.52e−5 | 7.68e−7 |
| Nle−Pro-Ile | 2.19e−5 | 5.27e−7 |
| Nva-Pro-Ile | 2.49e−5 | 8.23e−7 |
| Orn-Pro-Ile | 2.16e−4 | 4.44e−5 |
| Phe−Pro-Ile | 6.20e−5 | 2.74e−6 |
| Phg-Pro-Ile | 1.54e−4 | 1.34e−5 |
| Pip-Pro-Ile | >0.100 | |
| Ser(P)-Pro-Ile | 1.20e−2 | 0.0015 |
| Ser(Bzl)-Pro-Ile | 6.78e−5 | 3.07e−6 |
| Ser-Pro-Ile | 2.81e−4 | 4.69e−5 |
| t-butyl-Gly-Pro-D-Val | 1.12e−4 | 5.62e−6 |
| t-butyl-Gly-Pro-Gly | 5.63e−5 | 1.67e−6 |
| t-butyl-Gly-Pro-Ile | 9.34e−6 | 9.08e−7 |
| t-butyl-Gly-Pro-Ile-NH₂ | 2.29e−5 | 1.13e−6 |
| t-butyl-Gly-Pro-t-butyl-Gly | 2.45e−5 | 8.01e−7 |
| t-butyl-Gly-Pro-Val | 1.38e−5 | 1.28e−6 |
| Thr-Pro-Ile | 1.00e−4 | 4.43e−6 |
| Tic-Pro-Ile | 0.0008 | 9.28e−6 |
| Trp-Pro-Ile | 3.17e−4 | 1.80e−5 |
| Tyr(P)-Pro-Ile | 1.77e−3 | 9.36e−4 |
| Tyr-Pro-(allo)Ile | 6.41e−5 | 3.07e−6 |
| Val-Pro-(allo)Ile | 1.80e−5 | 7.61e−7 |
| Val-Pro-Val | 1.64e−5 | 1.22e−6 |

t-butyl-Gly is defined as:

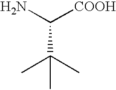

Ser(Bzl) and Ser(P) are defined as benzyl-serine and phosphoryl-serine, respectively. Tyr(P) is defined as phosphoryl-tyrosine.

TABLE 3
Kᵢ -values of Xaa-Pro-Yaa tripeptides

| Compound | Kᵢ (mol/l) | SD (mol/l) |
|---|---|---|
| Abu-Pro-Ile | 8.75e−6 | 1.52e−6 |
| AOA-Pro-Ile | 1.26e−5 | 2.2e−6 |
| Aze−Pro-Ile | 2.05e−5 | 3.77e−6 |
| Cha-Pro-Ile | 5.99e−6 | 2.11e−7 |
| Diprotin A | 3.45e−6 | 2.08e−7 |
| Diprotin B | 2.24e−5 | 1.5e−7 |
| Ile−Pro-(allo)Ile | 5.22e−6 | 2.58e−7 |
| Ile−Pro-t-butyl-Gly | 1.89e−5 | 8.30e−7 |
| Ile−Pro-Val | 5.25e−6 | 1.82e−8 |
| Nle−Pro-Ile | 9.60e−6 | 3.18e−8 |
| Nva-Pro-Ile | 6.17e−6 | 1.08e−6 |
| Phe−Pro-Ile | 1.47e−5 | 3.92e−8 |
| Ser(Bz)-Pro-Ile | 2.16e−5 | 1.79e−6 |
| t-butyl-Gly-Pro-D-Val | 2.65e−5 | 1.63e−7 |
| t-butyl-Gly-Pro-Gly | 1.51e−5 | 8.70e−7 |
| t-butyl-Gly-Pro-Ile | 3.10e−6 | 1.56e−8 |
| t-butyl-Gly-Pro-Ile−NH₂ | 5.60e−6 | 1.24e−8 |
| t-butyl-Gly-Pro-t-butyl-Gly | 1.41e−5 | 1.18e−7 |
| t-butyl-Gly-Pro-Val | 3.10e−6 | 1.60e−7 |
| Tyr-Pro-(allo)Ile | 1.82e−5 | 3.36e−8 |
| Val-Pro-(allo)Ile | 9.54e−6 | 2.56e−8 |
| Val-Pro-t-butyl-Gly | 1.96e−5 | 1.31e−6 |
| Val-Pro-Val | 4.45e−6 | 3.78e−9 |

t-butyl-Gly is defined as:

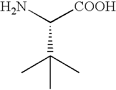

Ser(Bzl) and Ser(P) are defined as benzyl-serine and phosphoryl-serine, respectively. Tyr(P) is defined as phosphoryl-tyrosine.

### Example 3

### The Influence of Xaa-Pro-Yaa Tripeptides on Plasma Activity of DP IV After Intravasal and Oral Administration in Wistar Rats

### Study Design

Animals

N=10 male Wistar rats (Shoe:Wist(Sho)) with a body weight>350 g were purchased from Tierzucht Schönwalde (Schönwalde, Germany).

Housing Conditions

Animals were single-housed under conventional conditions with controlled temperature (22±2° C.) on a 12/12 hours light/dark cycle (light on at 06:00 AM). Standard pelleted chow (ssniff® Soest, Germany) and tap water acidified with HCl were allowed ad libitum.

Catheterization of Carotid Artery and Jugular Vein

After one week of adaptation to housing conditions, catheters were implanted into the carotid artery of Wistar rats under general anaesthesia (i.p. injection of 0.25 ml/kg b.w.

Rompun® [2%], BayerVital, Germany and 0.5 ml/kg b.w. Ketamin 10, Atarost GmbH & Co., Twistringen, Germany). The animals were allowed to recover for one week. The catheters were flushed with heparin-saline (100 IU/ml) three times per week.

In case of catheter dysfunction, a second catheter was inserted into the contra-lateral carotid artery of the respective rat. After one week of recovery from surgery, this animal was reintegrated into the study. In case of dysfunction of the second catheter, the animal was withdrawn from the study. A new animal was recruited and the experiments were continued in the planned sequence, beginning at least 5 days after catheter implantation.

Experimental Design

Rats with intact catheter function were given the test substances intravasal (intraarterial) or oral, respectively, in random order (N=3 Wistar rats in each group). As positive control, mg/kg b.w. isoleucine thiazolidine*fumarate were administered intravasal.

After overnight fasting, 100 μl samples of heparinized arterial blood were collected at −30, −5, and 0 min into ice-cooled Eppendorf tubes (see below). The test substances were dissolved freshly in 1.0 ml saline (0.154 mol/l) and were given at 0 min either oral via a feeding tube (15 g, 75 mm; Fine Science Tools, Heidelberg, Germany) or intravasal. For the intravasal route, the catheter was immediately flushed with 30 μl saline and an additional 1 ml of saline was given orally via the feeding tube.

Arterial blood samples were taken thereafter at 5, 10 (only in a limited number of experiments), 20, 40, 60 and 120 min from the carotid catheter of the conscious unrestrained rats and were always given into ice cooled Eppendorf tubes (Eppendorf-Netheler-Hinz, Hamburg, Germany) filled with 10 μl 1 M citrate buffer pH 3.0 for prevention of further hydrolysis of tripeptides by plasma DP IV activity. Eppendorf tubes were centrifuged immediately (12000 rpm for 2 min, Hettich Zentrifuge EBA 12, Tuttlingen; Germany): The plasma fractions were store on ice until analysis or were deeply frozen at −20 ° C. until analysis.

Analytical Methods

Plasma DP IV activity: The assay mixture consisted of 80 μl reagent and 20 μl plasma. Kinetic measurements of the formation of the yellow product 4-nitroaniline were performed at 390 nm for 1 min at 30° C. after 2 min pre-incubation at the same temperature. The activity was expressed as arbitrary units [AU] and DP IV activity [mU/ml].

Statistical Methods

The absolute values of plasma DP IV activity, the relative change of plasma DP IV activity and the time and extent of maximal inhibition were calculated. Data presentation included the presentation of area under the curve (AUC), which was used for evaluation of extent of inhibition of plasma DP IV activity during the two hours of observation. AUC was calculated using the trapezoidal rule. Reactive AUC had a baseline with the value at 0 min with beginning of the inhibitor administration.

To compare relative changes of parameters under the conditions of different initial values of DP IV activity an uniform standardised mean (values) was set up at the beginning of the test (see Figures and Tables).

Statistical evaluations were performed with Microsoft Excel® 97. All variables are presented as mean and standard deviation (SD). Treatment groups were compared by Student's t-test, within-group changes by paired t-test. Two-tailed values of p<0.05 were considered significant.

### Results

### The Influence of Ile-Pro-Ile On Plasma Dp IV Activity in Wistar-Rats

Ascending doses of Ile-Pro-Ile of 10, 30 and 100 mg/kg b.w. were administered to Wistar rats via intravasal route (Table 1).

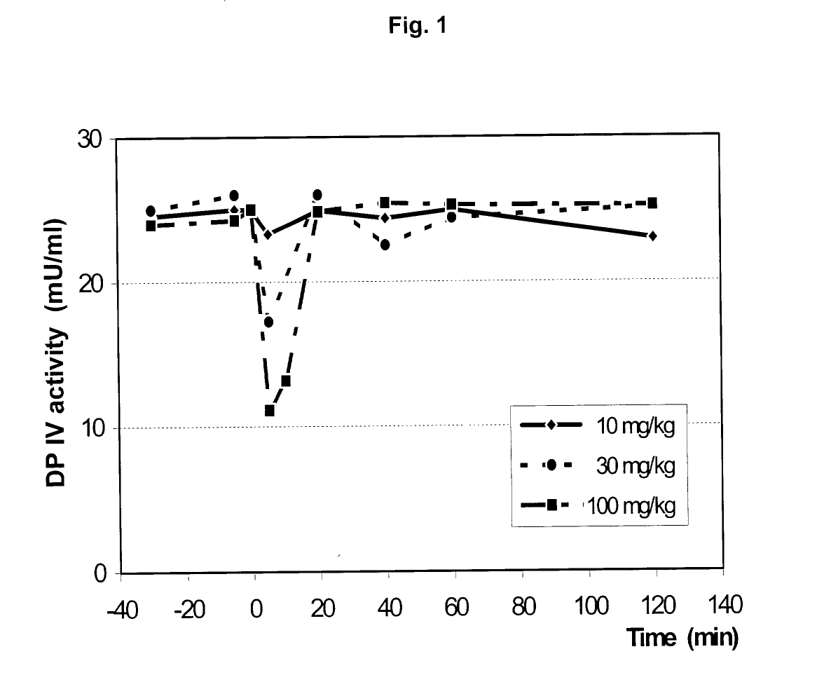

After intravasal administration of Ile-Pro-Ile there is a tendency of dose-dependent plasma DP IV inhibition by Ile-Pro-Ile. This inhibition was significant only at the dose of 100 mg/kg b.w. (with respect to DP IV activity at time 0 min). See FIG. 1 which shows that the inhibition of DP IV by Ile-Pro-Ile was decreased rapidly. 20 minutes after administration, the initial level of dipeptidyl peptidase IV activity was restored.

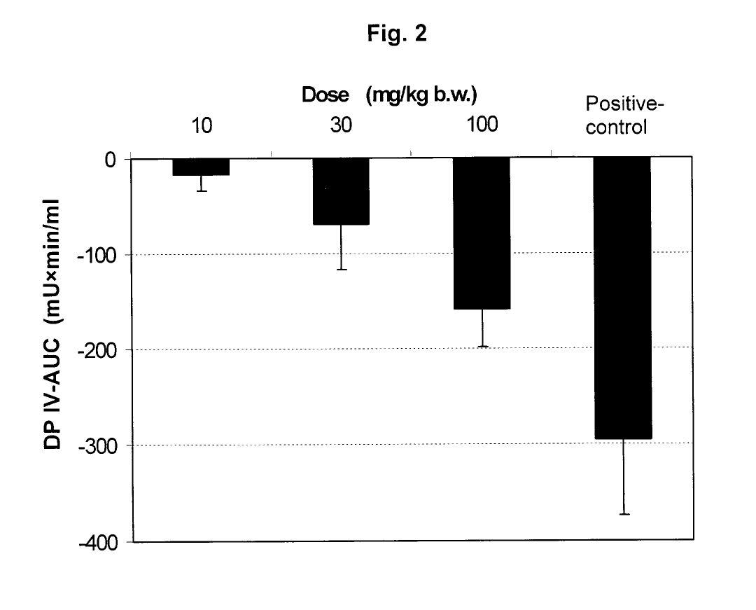

With reference to FIG. 2, it can be seen that the area under the curve of DP IV activity at the dose of 100 mg/kg b.w. was significantly decreased (−159±40 mU*min*ml⁻¹, p<0.05).

TABLE 4
Plasma DP IV activity of Wistar rats after
intravasal application of Ile-Pro-Ile.

| Ile-Pro-Ile | | | | | | | | | | | | | | | |
|---|---|---|---|---|---|---|---|---|---|---|---|---|---|---|---|
| intravasal | | | | | | | | | | | | | | AUC | |
| DP IV | | TIME | | | | | | | | | | Time of | | (mU · min/ml) | |
| (mU/ml) | Date | −30 | −5 | 0 | 5 | 10 | 20 | 40 | 60 | 120 | Minimum | Minimum | Drop | 0-120 | 0-20 |
| 10 mg/kg | | | | | | | | | | | | | | | |
| FS-5 | Apr 03 | 24.4 | 22.6 | 25.8 | 23.6 | | 24.2 | 24.3 | 24.3 | 24.8 | 23.6 | 5 | 2.1 | −171.4 | −33.7 |
| FS-2 | Apr 02 | 27.2 | 29.8 | 25.9 | 23.2 | | 27.2 | 25.4 | 28.5 | 23.2 | 23.2 | 5 | 2.6 | 14.3 | −16.4 |
| FS-7 | Apr 06 | 19.3 | 19.7 | 20.5 | 20.2 | | 20.6 | 20.6 | 19.3 | 18.0 | 20.2 | 5 | 0.3 | −122.5 | −2.4 |
| Mean | | 23.6 | 24.1 | 24.1 | 22.4 | | 24.0 | 23.5 | 24.0 | 22.0 | 22.4 | 5.0 | 1.7 | −93.2 | −17.5 |
| SD | | 4.0 | 5.2 | 3.1 | 1.9 | | 3.3 | 2.5 | 4.6 | 3.6 | 1.9 | 0.0 | 1.2 | 96.2 | 15.7 |
| Standard. | | 24.6 | 25.0 | 25.0 | 23.3 | | 24.9 | 24.4 | 25.0 | 22.9 | 22.9 | | | | |
| Mean | | | | | | | | | | | | | | | |
| 30 mg/kg | | | | | | | | | | | | | | | |
| FS-2 | Apr 10 | 32.0 | 34.6 | 30.3 | 25.0 | | 32.9 | 25.4 | 31.1 | 34.2 | 25.0 | 5 | 5.3 | 50.4 | −32.9 |
| FS-9 | Apr 11 | 25.0 | 25.0 | 28.1 | 15.4 | | 28.5 | 25.0 | 28.1 | 25.0 | 15.4 | 5 | 12.7 | −273.0 | −123.9 |
| FS-14 | Apr 26 | 16.7 | 17.1 | 15.4 | 10.1 | | 15.4 | 15.8 | 12.7 | 14.9 | 10.1 | 5 | 5.3 | −162.3 | −52.6 |
| Mean | | 24.6 | 25.6 | 24.6 | 16.8 | | 25.6 | 22.1 | 24.0 | 24.7 | 16.8 | 5.0 | 7.7 | −128.3 | −69.8 |
| SD | | 7.7 | 8.8 | 8.1 | 7.6 | | 9.1 | 5.4 | 9.9 | 9.7 | 7.6 | 0.0 | 4.3 | 164.4 | 47.9 |
| Standard. | | 25.0 | 26.0 | 25.0 | 17.3 | | 26.0 | 22.5 | 24.4 | 25.1 | 17.3 | | | | |
| Mean | | | | | | | | | | | | | | | |
| 100 mg/kg | | | | | | | | | | | | | | | |
| FS-13 | Apr 23 | 16.7 | 19.3 | 21.1 | 5.3 | 6.6 | 19.7 | 21.1 | 19.7 | 21.9 | 5.3 | 5 | 15.8 | −233.6 | −194.1 |
| FS-10 | Apr 30 | 21.9 | 20.6 | 21.1 | 4.8 | 8.8 | 22.4 | 22.8 | 20.2 | 20.0 | 4.8 | 5 | 16.2 | −185.1 | −166.7 |
| FS-14 | Apr 30 | 15.4 | 14.9 | 14.9 | 5.3 | 6.1 | 14.5 | 14.5 | 18.0 | 15.8 | 5.3 | 5 | 9.6 | 19.7 | −116.2 |
| Mean | | 18.0 | 18.3 | 19.0 | 5.1 | 7.2 | 18.9 | 19.4 | 19.3 | 19.2 | 5.1 | 5.0 | 13.9 | −133.0 | −159.0 |
| SD | | 3.5 | 3.0 | 3.5 | 0.3 | 1.4 | 4.0 | 4.4 | 1.2 | 3.1 | 0.3 | 0.0 | 3.7 | 134.4 | 39.5 |
| Standard. | | 24.0 | 24.3 | 25.0 | 11.1 | 13.2 | 24.9 | 25.4 | 25.3 | 25.2 | 11.1 | | | | |
| Mean | | | | | ∘ | ∘ | | | | | * | | * + | | * + |

### The Influence of Val-Pro-Leu on Plasma DP IV Activity in Wistar-Rats

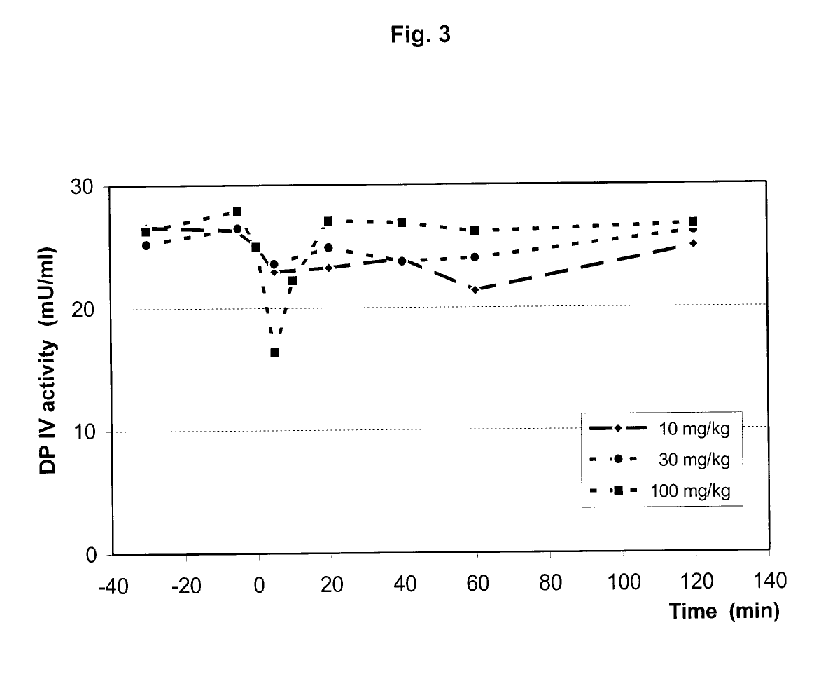

A tendency to decline plasma DP IV activity was seen after 10 and 30 mg/kg b.w. of Val-Pro-Leu given intravasal (FIG. 3). After 10 mg/kg b.w. lowest plasma activity was 26.2±8.0 mU/ml at 5 min (NS) and after 30 mg/kg it was 21.8±9.8 (NS). This was also reflected in the low DP IV-AUC_{0-20 min} of −34±6 mU·min·ml⁻¹ after 10 mg/kg b.w. and of −10±10 mU·min·ml⁻¹ after 30 mg/kg b.w. of Val-Pro-Leu (NS).

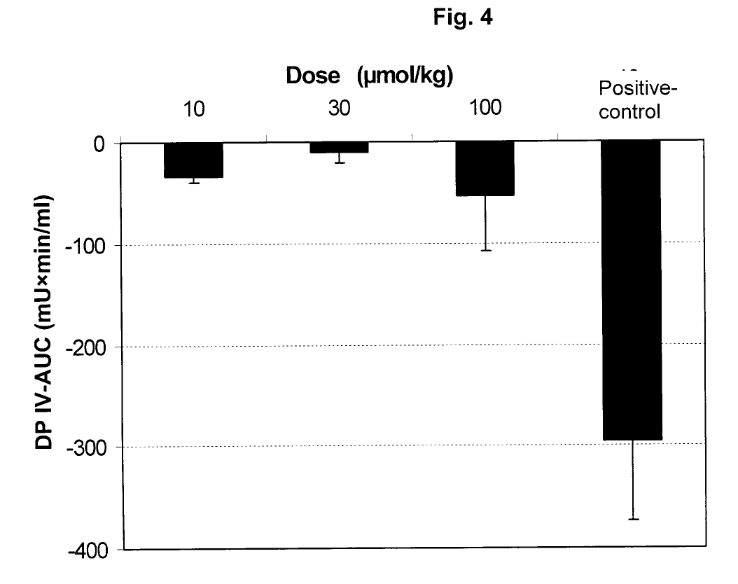

With reference to FIG. 4, 100 mg/kg b.w. declined plasma DP IV activity at 5 min (10.4±3.2 mU/ml; p<0.05 vs. 0 min) after intravasal administration and DP IV-AUC_{0-20 min} was therefore slightly declined (−54±54 mU·min·ml⁻¹; NS).

The inhibition of plasma DP IV was always stopped 20 min after the administration.

TABLE 5
Plasma DP IV activity of Wistar rats after
intravasal application of Val-Pro-Leu.

| Ile-Pro-Ile | | | | | | | | | | | | | | | |
|---|---|---|---|---|---|---|---|---|---|---|---|---|---|---|---|
| intravasal | | | | | | | | | | | | | | AUC | |
| DP IV | | TIME | | | | | | | | | | Time of | | (mU · min/ml) | |
| (mU/ml) | Date | −30 | −5 | 0 | 5 | 10 | 20 | 40 | 60 | 120 | Minimum | Minimum | Drop | 0-120 | 0-20 |
| 10 mg/kg | | | | | | | | | | | | | | | |
| FS-5 | Apr 03 | 24.4 | 22.6 | 25.8 | 23.6 | | 24.2 | 24.3 | 24.3 | 24.8 | 23.6 | 5 | 2.1 | −171.4 | −33.7 |
| FS-2 | Apr 02 | 27.2 | 29.8 | 25.9 | 23.2 | | 27.2 | 25.4 | 28.5 | 23.2 | 23.2 | 5 | 2.6 | 14.3 | −16.4 |
| FS-7 | Apr 06 | 19.3 | 19.7 | 20.5 | 20.2 | | 20.6 | 20.6 | 19.3 | 18.0 | 20.2 | 5 | 0.3 | −122.5 | −2.4 |
| Mean | | 23.6 | 24.1 | 24.1 | 22.4 | | 24.0 | 23.5 | 24.0 | 22.0 | 22.4 | 5.0 | 1.7 | −93.2 | −17.5 |
| SD | | 4.0 | 5.2 | 3.1 | 1.9 | | 3.3 | 2.5 | 4.6 | 3.6 | 1.9 | 0.0 | 1.2 | 96.2 | 15.7 |
| Standard. | | 24.6 | 25.0 | 25.0 | 23.3 | | 24.9 | 24.4 | 25.0 | 22.9 | 22.9 | | | | |
| Mean | | | | | | | | | | | | | | | |
| 30 mg/kg | | | | | | | | | | | | | | | |
| FS-2 | Apr 10 | 32.0 | 34.6 | 30.3 | 25.0 | | 32.9 | 25.4 | 31.1 | 34.2 | 25.0 | 5 | 5.3 | 50.4 | −32.9 |
| FS-9 | Apr 11 | 25.0 | 25.0 | 28.1 | 15.4 | | 28.5 | 25.0 | 28.1 | 25.0 | 15.4 | 5 | 12.7 | −273.0 | −123.9 |
| FS-14 | Apr 26 | 16.7 | 17.1 | 15.4 | 10.1 | | 15.4 | 15.8 | 12.7 | 14.9 | 10.1 | 5 | 5.3 | −162.3 | −52.6 |
| Mean | | 24.6 | 25.6 | 24.6 | 16.8 | | 25.6 | 22.1 | 24.0 | 24.7 | 16.8 | 5.0 | 7.7 | −128.3 | −69.8 |
| SD | | 7.7 | 8.8 | 8.1 | 7.6 | | 9.1 | 5.4 | 9.9 | 9.7 | 7.6 | 0.0 | 4.3 | 164.4 | 47.9 |
| Standard. | | 25.0 | 26.0 | 25.0 | 17.3 | | 26.0 | 22.5 | 24.4 | 25.1 | 17.3 | | | | |
| Mean | | | | | | | | | | | | | | | |
| 100mg/kg | | | | | | | | | | | | | | | |
| FS-13 | Apr 23 | 16.7 | 19.3 | 21.1 | 5.3 | 6.6 | 19.7 | 21.1 | 19.7 | 21.9 | 5.3 | 5 | 15.8 | −233.6 | −194.1 |
| FS-10 | Apr 30 | 21.9 | 20.6 | 21.1 | 4.8 | 8.8 | 22.4 | 22.8 | 20.2 | 20.0 | 4.8 | 5 | 16.2 | −185.1 | −166.7 |
| FS-14 | Apr 30 | 15.4 | 14.9 | 14.9 | 5.3 | 6.1 | 14.5 | 14.5 | 18.0 | 15.8 | 5.3 | 5 | 9.6 | 19.7 | −116.2 |
| Mean | | 18.0 | 18.3 | 19.0 | 5.1 | 7.2 | 18.9 | 19.4 | 19.3 | 19.2 | 5.1 | 5.0 | 13.9 | −133.0 | −159.0 |
| SD | | 3.5 | 3.0 | 3.5 | 0.3 | 1.4 | 4.0 | 4.4 | 1.2 | 3.1 | 0.3 | 0.0 | 3.7 | 134.4 | 39.5 |
| Standard. | | 24.0 | 24.3 | 25.0 | 11.1 | 13.2 | 24.9 | 25.4 | 25.3 | 25.2 | 11.1 | | | | |
| Mean | | | | | ∘ | ∘ | | | | | * | | * + | | * + |

The compounds Ile-Pro-Ile and Val-Pro-Leu inhibit plasma DP IV-activity in Wistar rats after intravasal administration in relatively high doses (100 mg/kg b.w.). With Ile-Pro-Ile it seems to be a dose dependent inhibition of DP IV after intravasal administration.

### The Influence of T-Butyl-Gly-Pro-Ile On Plasma Dp Iv Activity in Wistar-Rats

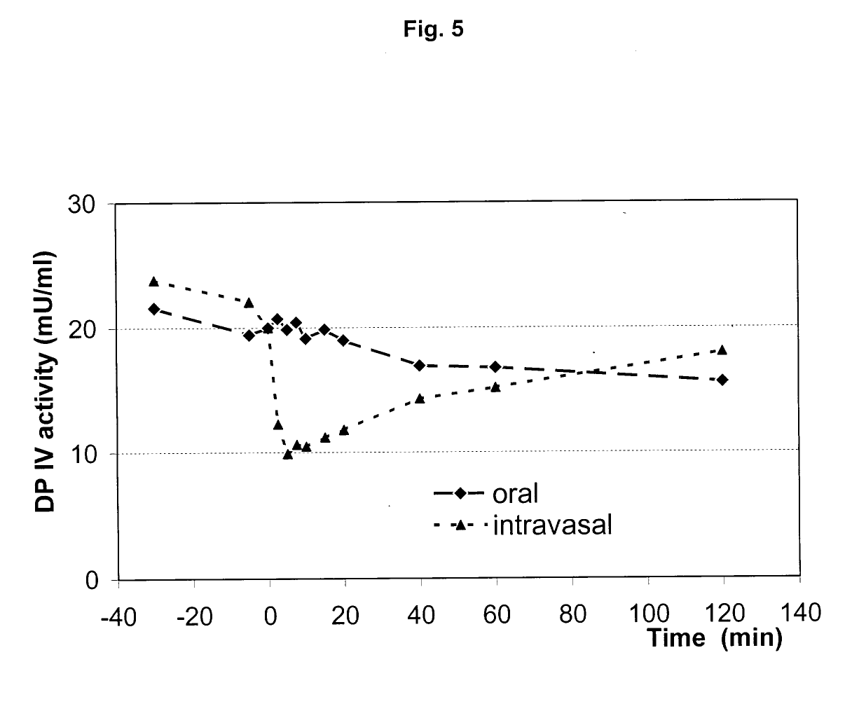

100 mg/kg b.w. t-butyl-Gly-Pro-Ile was administered oral and intravasal. With reference to FIG. 5, t-butyl-Gly-Pro-Ile did decrease plasma DP IV slowly over a time period of 40 min when given orally. 100 mg/kg b.w. given intravasal, induced a very rapid decline below 10 mU/ml at 5 min (p<0.05). Thereafter a restoration (p<0.05 5 min vs. 40 min) was found. DP IV-AUC_{0-120 min} was more declined after intra-vasal (−617±234 mU·min·ml⁻¹) than after oral administration (−336±162 mU·min·ml⁻¹; p<0.05 vs. intra-vasal).

TABLE 6
Plasma DP IV activity after oral administration of t-butyl-Gly-Pro-Ile in Wistar rats.

| t-butyl-Gly-Pro-Ile, | | | | | | | | | | | | | | | DP IV- |
|---|---|---|---|---|---|---|---|---|---|---|---|---|---|---|---|
| oral | TIME | | | | | | | | | | | | | | AUC |
| DP IV (mU/ml) | −30 | −5 | 0 | 2.5 | 5 | 7.5 | 10 | 15 | 20 | 40 | 60 | 120 | Min. | Drop | (AU · min) |
| | 21.93 | 23.68 | 19.74 | 20.61 | 23.68 | 21.05 | 21.93 | 29.39 | 25.00 | 21.49 | 21.93 | 16.23 | 16.23 | 6.58 | 155.2 |
| 53-7 | 26.75 | 37.28 | 23.68 | 9.21 | 13.16 | 12.28 | 25.88 | 15.79 | 10.97 | 8.77 | 14.47 | 23.25 | 8.77 | 23.25 | −961.1 |
| 53-10 | 18.86 | 18.86 | 17.11 | 21.93 | 20.61 | 18.42 | 17.11 | 17.98 | | 27.63 | 21.93 | 23.25 | 17.11 | 1.75 | 651.3 |
| Mean | 22.51 | 26.61 | 20.18 | 17.25 | 19.15 | 17.25 | 21.64 | 21.05 | 17.98 | 19.30 | 19.44 | 20.91 | 14.04 | 10.53 | −51.5 |
| SD | 3.98 | 9.55 | 3.31 | 6.99 | 5.41 | 4.50 | 4.39 | 7.30 | 9.92 | 9.62 | 4.30 | 4.05 | 4.58 | 11.28 | 825.8 |
| Standardized Mean | 22.34 | 26.43 | 20.00 | 17.08 | 18.98 | 17.08 | 21.46 | 20.88 | 17.81 | 19.12 | 19.27 | 20.73 | 17.08 | | |

TABLE 7
Plasma DP IV activity after intravasal administration of t-butyl-Gly-Pro-Ile in Wistar rats.

| t-butyl-Gly-Pro-Ile, iv | TIME | | | | | | | | | | | | | | DP IV-AUC |
|---|---|---|---|---|---|---|---|---|---|---|---|---|---|---|---|
| DP IV (mU/ml) | −30 | −5 | 0 | 2.5 | 5 | 7.5 | 10 | 15 | 20 | 40 | 60 | 120 | Min. | Drop | (AU · min) |
| 53-4 | 19.30 | 21.05 | 19.74 | 15.35 | 15.35 | 17.11 | 17.11 | 14.47 | 15.35 | 14.91 | 15.79 | 17.11 | 14.47 | 5.70 | −452.9 |
| 53-7 | 24.12 | 25.88 | 33.77 | 27.63 | 25.00 | 27.63 | 20.18 | 18.42 | 25.44 | 23.68 | 22.37 | 25.88 | 18.42 | 6.58 | −1179.3 |
| 53-10 | 17.11 | 16.67 | 17.98 | 14.04 | 16.23 | 18.42 | 14.47 | 13.60 | 15.35 | 15.35 | 13.60 | 15.35 | 13.60 | 3.29 | −388.2 |
| Mean | 20.18 | 21.20 | 23.83 | 19.01 | 18.86 | 21.05 | 17.25 | 15.50 | 18.71 | 17.98 | 17.25 | 19.44 | 15.50 | 5.19 | −673.4 |
| SD | 3.59 | 4.61 | 8.65 | 7.50 | 5.34 | 5.74 | 2.85 | 2.57 | 5.82 | 4.94 | 4.57 | 5.64 | 2.57 | 1.70 | 439.3 |
| Standardized Mean | 16.35 | 17.37 | 20.00 | 15.18 | 15.03 | 17.22 | 13.42 | 11.67 | 14.88 | 14.15 | 13.42 | 15.61 | 11.67 | | |

Table 8 shows the results of selected Xaa-Pro-Yaa tripeptides, tested for their inhibitory potential of DPIV and DPIV-like enzyme activity after oral and intravasal administration to Wistar rats.

TABLE 8
Results - DRIV inhibiton at tₘₐₓ after
administration of Xaa-Pro-Yaa
tripeptides to Wistar rats

| | Dose | | |
|---|---|---|---|
| Structure | (mg/kg) | i.v. (%) | p.o. (%) |
| Diprotin A (Ile−Pro-Ile) | 100 | 73 | no inhibition |
| Diprotin B (Val-Pro-Leu) | 100 | 50 | no inhibition |
| Tyr(P)-Pro-Ile | 100 | 37 | no inhibition |
| t-butyl-Gly-Pro-Ile | 100 | 71 | 28 |
| t-butyl-Gly-Pro-Val | 100 | 72 | 25 |

### Example 4

### The Effect of Xaa-Pro-Yaa Tripeptides on Glucose Tolerance in Diabetic Zucker Rats

### Study Design

Animals

N=30 male Zucker rats (fa/fa), mean age 11 weeks (5-12 weeks), mean body weight 350 g (150-400 g), were purchased from Charles River (Sulzfeld, Germany). They were kept for >12 weeks until all the fatty Zucker rats had the characteristics of manifest Diabetes mellitus.

Housing Conditions

Animals were kept single-housed under conventional conditions with controlled temperature (22±2° C.) on a 12/12 hours light/dark cycle (light on at 06:00 a.m.). Standard pellets (ssniff®, Soest, Germany) and tap water acidified with HCl were allowed ad libitum.

Catheterization Of Carotid Artery

Fatty Zucker rats, 17-24 weeks old, adapted to the housing conditions, were well prepared for the tests. Catheters were implanted into the carotid artery of fatty Zucker rats under general anaesthesia (i.p. injection of 0.25 ml/kg b.w. Rompun® [2 %], BayerVital, Germany and 0.5 ml/kg b.w. Ketamin 10, Atarost GmbH & Co., Twistringen, Germany). The animals were allowed to recover for one week. The catheters were flushed with heparin-saline (100 IU/ml) three times per week.

In case of catheter dysfunction, a second catheter was inserted into the contra-lateral carotid artery of the respective rat. After one week of recovery from surgery, this animal was reintegrated into the study. In case of dysfunction of the second catheter, the animal was withdrawn from the study. A new animal was recruited and the experiments were continued in the planned sequence, beginning at least 7 days after catheter implantation.

Experimental Design

Fatty Zucker rats with intact catheter function were given in random order placebo (1 ml saline, 0.154 mol/l; N=9 animals as control), one uniform dose of isoleucyl thiazolidine*fumarate (10 mg/kg b.w. solved in 1 ml saline; N=6 animals) as positive control or 100 mg/kg b.w. test substance, solved in 1 ml saline (N=6 animals in each test group).

After overnight fasting, the fatty Zucker rats were given placebo, positive control and test substance, respectively, via feeding tube orally (15 G, 75 mm; Fine Science Tools, Heidelberg, Germany) at −10 min. An oral glucose tolerance test (OGTT) with 2 g/kg b.w. glucose as a 40% solution (B. Braun Melsungen, Melsungen, Germany) was implemented at ±0 min. The glucose was administered via a second feeding tube. Arterial blood samples from the carotid catheter were collected at −30 min, −15 min, ±0 min and at 5, 10, 15, 20, 30, 40, 60, 90 and 120 min into 20 μl glass capillaries, which were placed in standard tubes filled with 1 ml solution for hemolysis (blood glucose measurement).

In addition, arterial blood samples were taken at −30 min, at 20, 40 60 and 120 min from the carotid catheter of the conscious unrestrained fatty Zucker rats and given into ice cooled Eppendorf tubes (Eppendorf-Netheler-Hinz, Hamburg, Germany) filled with 10 μl sodium citrate buffer (pH 3.0) for plasma DP activity measurement. Eppendorf tubes were centrifuged immediately (12000 rpm for 2 min, Hettich Zentrifuge EBA 12, Tuttlingen; Germany): The plasma fractions were stored on ice until analysis.

Analytical Methods

Blood glucose: Glucose levels were measured using the glucose oxidase procedure (Super G Glukosemeβgerät; Dr. Müller Gerätebau, Freital, Germany).

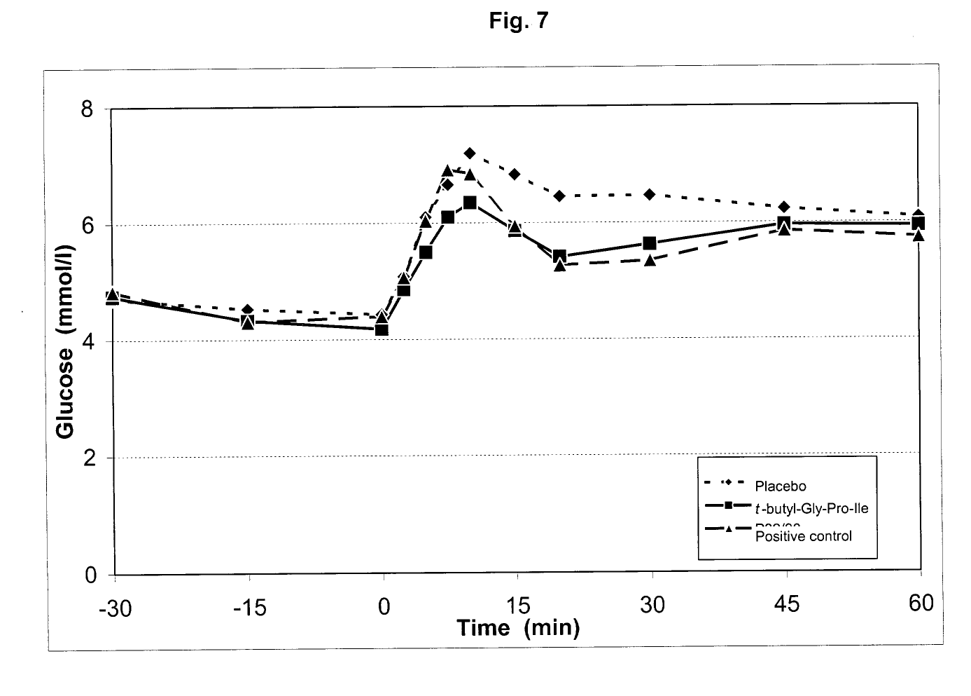

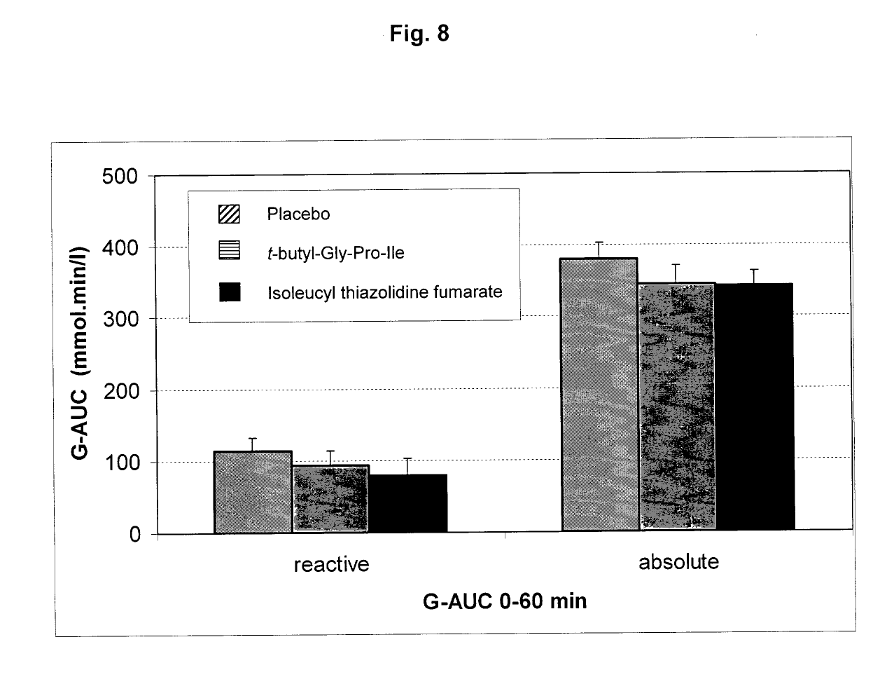

Several tripeptides, tested in the in vivo assay according to example 4, improved significantly the glucose tolerance after oral administration during an OGTT in Zucker rats (see table 9 and FIGS. 7 and 8).

TABLE 9
Results - Improvement of glucose tolerance after administration of Xaa-
Pro-Yaa tripeptides during an OGTT in Zucker rats

| | Dose | | | AUC test | |
|---|---|---|---|---|---|
| | (mg/kg | Route | AUC Control | compound | Improvement |
| Compound | b.w.) | of adm. | (mmol * min/l) | (mmol * min/l) | % |
| t-butyl-Gly-Pro-Ile | 100 | p.o. | 766.2 | 653.2 | 14.8 |
| Val-Pro-t-butyl-Gly | 100 | p.o. | 865.6 | 722.4 | 16.5 |
| Ile-Pro-t-butyl-Gly | 100 | p.o. | 865.6 | 819.5 | 5.3 |

### EXAMPLE 5

### Interaction of Peptidic Compounds with Mammalian Peptide Transporters

For the analysis o the interaction of the inhibitors of prolyl-specific proteases with the mammalian peptide transporters two assay systems were used. First, all test compounds were submitted to the competition assay with transgenic yeast cells to determine the dose-dependent displacement (EC₅₀ values) of the radiolabeled tracer dipepitde from the substrate binding site. Compounds identified as possessing good affinities were then submitted to electrophysiological analysis of transport currents in Xenopus oocytes expressing the mammalian peptide transporters. As the level of functional expression varies in oocytes, each test compound was compared in the same oocyte with the current elicited by 5 mM of the dipeptide glycyl-L-glutamine (Gly-Gln). Currents of test compounds are therefore given relative to that by Gly-Gln as %I _{Gly-Gin}, expressed as residual uptake in table 10.

TABLE 10
Results - Interaction of peptidic compounds with
mammalian peptide transporters

| | | PEPT1 | | PEPT2 |
|---|---|---|---|---|
| | | Residual | | Residual |
| | PEPT1 | uptake | PEPT2 | uptake |
| Substance | EC₅₀ (mM) | % I_{Gly-Gln} | EC₅₀ (mM) | % I_{Gly-Gln} |
| Cha-Pro-Ile | 0.141 ± 0.009 | 90 | 0.124 ± 0.001 | 100 |
| Tyr(P)-Pro-Ile | 0.902 ± 0.058 | 0 | 0.390 ± 0.017 | 0 |
| Ser(P)-Pro-Ile | 4.1 ± 1.3 | 0 | 4.9 ± 1.09 | 0 |
| t-butyl-Gly- | 11.1 ± 2.8 | 0 | 1.5 ± 0.24 | 0 |
| Pro-D-Val | | | | |
| t-butyl-Gly- | 10.6 ± 0.064 | 0 | 2.3 ± 1.12 | 0 |
| Pro-D-Ile | | | | |
| Ile−Pro-Val | 0.319 ± 0.04 | 65 | 0.172 ± 0.03 | 200 |

While the foregoing specifications teaches the principles of the present invention, with examples provided for the purpose of illustration, it will be understood that the practice of the invention encompasses all of the usual variations, adaptations and/or modifications envisioned by one of ordinary skill in the art.

1. A compound represented by the general formula (I): 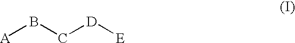
and pharmaceutically acceptable salts thereof,
wherein
A is any amino acid except a D-amino acid;
B is an amino acid selected from Pro, Ala, Ser, Gly, Hyp, acetidine-(2)-carboxylic acid and pipecolic acid,
C is any amino acid except Pro, Hyp, acetidine-(2)-carboxylic acid, pipecolic acid and except N-alkylated amino acids, e.g. N-methyl valine and sarcosine,
D is any amino acid or missing, and
E is any amino acid or missing;
or
wherein
C is any amino acid except Pro, Hyp, acetidine-(2)-carboxylic acid, pipecolic acid, except N-alkylated amino acids, e.g. N-methyl valine and sarcosine and except a
D-amino acid,
D is an amino acid selected from Pro, Ala, Ser, Gly, Hyp, acetidine-(2)-carboxylic acid and pipecolic acid, and
E is any amino acid except Pro, Hyp, acetidine-(2)-carboxylic acid, pipecolic acid and except N-alkylated amino acids, e.g. N-methyl valine and sarcosine.

2. The compound of claim 1, wherein
A is any amino acid except a D-amino acid;
B is an amino acid selected from Pro, Ala, Ser, Gly, Hyp, acetidine-(2)-carboxylic acid and pipecolic acid,
C is any amino acid except Pro, Hyp, acetidine-(2)-carboxylic acid, pipecolic acid and except N-alkylated amino acids, e.g. N-methyl valine and sarcosine,
D is any amino acid or missing, and
E is any amino acid or missing.

3. The compound of claim 1, wherein
A is any amino acid except a D-amino acid;
B is an amino acid selected from Pro, Ala, Ser, Gly, Hyp, acetidine-(2)-carboxylic acid and pipecolic acid,
C is any amino acid except Pro, Hyp, acetidine-(2)-carboxylic acid, pipecolic acid, except N-alkylated amino acids, e.g. N-methyl valine and sarcosine and except a D-amino acid,,
D is an amino acid selected from Pro, Ala, Ser, Gly, Hyp, acetidine-(2)-carboxylic acid and pipecolic acid, and
E is any amino acid except Pro, Hyp, acetidine-(2)-carboxylic acid, pipecolic acid and except N-alkylated amino acids, e.g. N-methyl valine and sarcosine. .

4. The compound of any one of the preceding claims, wherein
A is a L-amino acid.

5. The compound of any one of the preceding claims, wherein
C is a L-amino acid.

6. The compound of any one of the preceding claims, wherein
E is missing.

7. The compound of any one of the preceding claims, wherein
D and E are missing.

8. The compound of any one of the preceding claims, wherein
A is t-butyl-Gly, lie or Val.

9. The compound of any one of the preceding claims, wherein
B is Pro.

10. The compound of any one of the preceding claims, wherein
C is t-butyl-Gly, lie or Val.

11. The compound of any one of the preceding claims, wherein
D is Pro, Ala, Ser, Gly, Hyp, acetidine-(2)-carboxylic acid or pipecolic acid.

12. The compound of claim 1, namely t-butyl-Gly-Pro-Ile; t-butyl-Gly-Pro-Val; Val-Pro- t-butyl-Gly, Ile-Pro-t-butyl-Gly or t-butyl-Gly-Pro-t-butyl-Gly and pharmaceuticaly acceptable salts thereof.

13. The compound of any one of the preceding claims, wherein the compound is the free acid peptide form or the C-terminal amide peptide form.

14. The compound of claim 13, wherein the free acid peptide form or the C-terminal amide peptide form is varied by side chain modifications selected from homoserine addition, pyroglutamic acid addition, disulphide bond formation, deamidation of asparagine or glutamine residues, methylation, t-butylation, t-butyloxycarbonylation, 4-methylbenzylation, thioanysilation, thiocresylation, benzyloxymethylation, 4-nitrophenylation, benzyloxycarbonylation, 2-nitrobenzoylation, 2-nitrosulphenylation, 4-toluenesulphonylation, pentafluorophenylation, diphenylmethylation, 2-chlorobenzyloxycarbonylation, 2,4,5-trichlorophenylation, 2-bromobenzyloxycarbonylation, 9-fluorenylmethyloxycarbonylation, triphenylmethylation, 2,2,5,7,8,-penta-methylchroman-6-sulphonylation, hydroxylation, oxidation of methionine, formylation, acetylation, anisylation, benzylation, benzoylation, trifluoroacetylation, carboxylation of aspartic acid or glutamic acid, phosphorylation, sulphation, cysteinylation, glycolysation with pentoses, deoxyhexoses, hexosamines, hexoses or N-acetylhexosamines, farnesylation, myristolysation, biotinylation, palmitoylation, stearoylation, geranylgeranylation, glutathionylation, 5′-adenosylation, ADP-ribosylation, modification with N-glycolylneuraminic acid, N-acetylneuraminic acid, pyridoxal phosphate, lipoic acid, 4′-phosphopantetheine, and N-hydroxysuccinimide.

15. Prodrugs of a compound of any one of the preceding claims.

16. A pharmaceutical composition comprising at least one compound or prodrug of any one of claims 1 to 15 and a pharmaceutically acceptable carrier and/or diluent.

17. A process for making a pharmaceutical composition comprising mixing at least one compound or a prodrug of any one of claims 1 to 15 and a pharmaceutically acceptable carrier and/or diluent.

18. Use of a compound, a prodrug or a composition according to any one of the preceding claims 1 to 15 for the preparation of a medicament for the prophylaxis or treatment of a condition mediated by modulation of the dipeptidyl peptidase IV activity.

19. Use of claim 17, wherein the condition is selected from impaired glucose tolerance, diabetes mellitus, glucosuria and metabolic acidosis.
